SEQUENCE LISTING (1 sequence)

SEQ ID NO: 1 - RNA, 22 nt, Homo sapiens
  misc_feature (1)..(22): microRNA-126
ucguaccgugaguaauaaugcg

(19) United States
(12) Patent Application Publication
(10) Pub. No.: US 2020/0297779 A1
(43) Pub. Date: Sep. 24, 2020

(54) METHODS, SYSTEMS, AND COMPOSITIONS RELATING TO TREATMENT OF NEUROLOGICAL CONDITIONS, DISEASES, AND INJURIES AND COMPLICATIONS FROM DIABETES

(71) Applicant: Henry Ford Health System, Detroit, MI (US)

(72) Inventors: Jieli CHEN, Troy, MI (US); Michael CHOPP, Southfield, MI (US)

(21) Appl. No.: 16/886,034

(22) Filed: May 28, 2020

Related U.S. Application Data
(62) Division of application No. 14/861,509, filed on Sep. 22, 2015.
(60) Provisional application No. 62/053,461, filed on Sep. 22, 2014.

(51) Int. Cl. A61K 35/51; C12N 15/113; A61K 35/44

(52) U.S. Cl. CPC A61K 35/51; C12N 2310/141; A61K 35/44; C12N 15/113

(57) ABSTRACT

Some embodiments comprise methods, systems, and/or compositions comprising the production and/or use of one or agents selected from a group comprising microRNA-126, a promoter of microRNA-126 expression, a microRNA-126 mimic, cells such as human umbilical cord blood cells (“HUCBCs”), endothelial cells (“EC”), endothelial progenitor cells (“EPC”), and microRNA-126-enriched Exosomes/Microvesicles (“EMVs”) to promote, increase, or improve recovery from conditions, diseases, or injuries and/or function or outcome in a patient in need thereof.

### CROSS-REFERENCE TO RELATED APPLICATIONS

This application is a Divisional of U.S. patent application Ser. No. 14/861,509, filed Sep. 22, 2015, which claims the benefit of U.S. Provisional Patent Application Ser. No. 62/053,461, filed Sep. 22, 2014. The contents of each of the aforementioned patent applications are incorporated herein by reference in their entireties.

### SEQUENCE LISTING

The instant application contains a Sequence Listing which has been submitted in ASCII format via EFS-Web and is hereby incorporated by reference in its entirety. Said ASCII copy, created on May 27, 2020, is named “NEUX-003_D01US_SeqList.txt” and is about 682 bytes in size.

### TECHNICAL FIELD

Without limitation, some embodiments comprise methods, systems, and/or compositions relating to microRNAs and/or cell-based therapies and the use of same in the research, diagnosis, or treatment of injury or disease.

### BACKGROUND

MicroRNAs (also “miRNAs” or “miRs”) are small non-coding sequences of RNA that have the capacity to regulate many genes, pathways, and complex biological networks within cells or tissues, acting either alone or in concert with one another. A need remains for therapeutic treatments of conditions, diseases, or injuries of mammalian subjects, including human beings, based in effective miRNA-based therapies and/or cell-based therapies.

### BRIEF DESCRIPTION OF THE DRAWINGS

Some embodiments will now be described, by way of example only and without waiver or disclaimer of other embodiments, with reference to the accompanying drawings, in which:

FIG. 1 is a data representation showing results of testing of HUCBC treatment of stroke on functional outcome.

FIG. 2A and FIG. 2B are data representation showing results of testing for miR-126 expression. FIG. 2A shows miR-126 expression in blood serum and FIG. 2B shows miR-126 expression in brain tissue.

FIG. 3A, FIG. 3B, FIG. 3C and FIG. 3D are data representations showing results of testing for miR-126 expression and functional outcome measurements. FIG. 3A and FIG. 3B show miR-126 expression and FIG. 3C and FIG. 3D shows functional tests.

FIG. 4A, FIG. 4B, FIG. 4C and FIG. 4D are data representations and images showing results of testing for miR-126 expression and capillary tube formation. FIG. 4A shows miR expression in BECs. FIG. 4B shows tube formation quantitative data. FIG. 4C and FIG. 4D show tube formation.

FIG. 5A, FIG. 5B, FIG. 5C, FIG. 5D, FIG. 5E, FIG. 5F and FIG. 5G are data representations and images showing results of testing for axonal outgrowth. FIG. 5A shows a microfluidic chamber culture. FIG. 5B, FIG. 5C, FIG. 5D, FIG. 5E, FIG. 5F show axonal outgrowth. FIG. 5G shows the quantitative data.

FIG. 6A, FIG. 6B, FIG. 6C and FIG. 6D are data representations showing results of testing for effects of miR-126 on Ang1 expression.

FIG. 7A and FIG. 7B are data representation showing results of testing for miR-126 expression. FIG. 7A shows miR-126 in HUCBC and EMVs and FIG. 7B shows miR-126 in BECs (B).

FIG. 8 is a data representation showing results of testing for miR-126 expression.

FIG. 9A and FIG. 9B are data representations showing results of testing for neurological functional outcome measured by Foot-fault and adhesive removal tests.

FIG. 10 is a data representation showing results of testing for cognitive functional outcome.

FIG. 11 is images and a data representation regarding results of testing for axonal outgrowth.

FIG. 12 is a data representation showing results of testing for OPC survival or proliferation.

### DETAILED DESCRIPTION

Without limitation to only those embodiments expressly disclosed herein, and without waiver or disclaimer of any embodiments or subject matter, some embodiments comprise methods, systems and/or compositions comprised of microRNA-126, microRNA-126 promoters or mimics, cells such as human umbilical cord blood cells (“HUCBCs”), and/or microRNA-126-enriched Exosomes/Microvesicles (“EMVs”) to promote neurovascular and white matter remodeling and induce neuroprotection and neurorestorative effects after stroke, neural injury (including without limitation, brain injury), multiple sclerosis, and dementia, neurodegenerative disease, and to ameliorate diabetes complications, in mammalian subjects, including without limitation, in human beings.

In general summary, we have discovered unexpectedly that, in accordance with some nonlimiting embodiments, microRNA-126, microRNA-126 promoters or mimics, cells such as human umbilical cord blood cells, and microRNA-126-enriched EMVs promote neurovascular and white matter remodeling and induce neuroprotection and neurorestorative effects after stroke, neural injury (including without limitation, brain injury), multiple sclerosis (“MS”), and dementia, neurodegenerative disease, and ameliorate diabetes complications.

MicroRNAs (also “miRNAs” or “miR's) are small non-coding sequences of RNA that have the capacity to regulate many genes, pathways, and complex biological networks within cells or tissues, acting either alone or in concert with one another. Certain miRNAs may be key players in the pathogenesis of type two diabetes (“T2DM”) and hyperglycemia-induced vascular damage. Among the miRNAs most consistently associated with diabetes (“DM”), is microRNA-126 (also “miR-126”) (e.g., SEQ ID No. 1, UCGUACCGUGAGUAAUAAUGCG). MiR-126 facilitates angiogenesis and regulates endothelial cell function. MiR-126 level is significantly decreased in the circulating vesicles in plasma of DM patients and in CD34+ peripheral blood mononuclear cell (“PBMCs”) in DM patients. In addition, miR-126 enhances the activities of Angiopoietin-1 (“Ang1”) on vessel stabilization and maturation by targeting p85beta. Our data indicate that mice with T2DM have significantly decreased blood serum and brain miR-126 and Ang1 expression after stroke compared to non-DM mice. We have found that treatment of stroke in T2DM mice with HUCBCs starting 3 days after ischemic stroke significantly increases ischemic brain tissue and blood serum miR-126, as well as improves functional outcome after stroke compared to non-treatment T2DM control mice. MiR-126 not only regulates vascular remodeling, but also promotes axonal outgrowth in cultured primary cortical neurons (“PCN”). Thus, some nonlimiting embodiments comprise a highly novel and clinically relevant approach to brain and vascular plasticity relating to the neurorestorative actions of miR-126. Our data indicate that generation of miR-126 contributes to its robust therapeutic effects; miR-126 promotes neurovascular and white matter (“WM”) remodeling, and thereby may induce neuroprotection and neurorestorative effects in diabetes, stroke, brain injury, dementia, MS and neurodegenerative diseases.

Some embodiments comprise use of miRNA, including without limitation, miR-126, in cell-based therapy. By post-transcriptionally affecting gene regulation, miRNAs are involved in most biological processes and act as molecular rheostats that fine-tune and switch regulatory circuits governing tissue repair, inflammation, hypoxia-response, and angiogenesis. As fine tools enabling specific and temporally controlled manipulation of cell-specific miRNAs, miRNA-based therapies may be effective in facilitating tissue repair. However, in vivo delivery of naked DNA, oligonucleotides, and miRNAs are complicated by their low stability, rapid degradation and inefficient delivery into target cells. Manipulation of miRNA expression with cell-based therapy has lower barriers, because cells can be delivered by intravenous injection and delivered cells continually release EMVs containing miRNA that stimulate endogenous brain plasticity. EMVs may not be mere byproducts resulting from cell activation or apoptosis. Instead, EMVs are enriched with nucleic acids (e.g., mRNA and miRNA). EMVs are secreted into the extracellular space and can be taken up by other cells. EMVs are biological vehicles for the transfer of nucleic acids and subsequently modulate the target cell's protein synthesis; thereby, they constitute a novel type of cell—cell mechanism of communication. Manipulation of miRNA expression in cell-based therapy and cell secretion of EMVs containing miRNA may further stimulate endogenous brain cells such as brain endothelia cells (“BECs”) or astrocytes to generate miR-126 or other miRNAs. We have found unexpectedly that HUCBCs secrete EMVs containing high level of miR-126, which increases BEC miR-126 expression. EMVs regulate the communication of miR-126 between brain BECs and neural cells, and thereby may promote vascular and WM remodeling. Thus, some nonlimiting embodiments comprise important and novel ways to understand how exogenously administered cells communicate with and alter endogenous brain cells by means of delivery miRNA to activate endogenous restorative events.

In accordance with some nonlimiting embodiments, miRNA, as only one example, miR-126, is delivered to increase vascular and WM remodeling, decrease inflammatory effects, and thereby reduce neurological deficits after stroke, neural trauma, multiple sclerosis, dementia and neurodegenerative disease and ameliorate diabetes complications. Much effort is underway to develop therapies which remodel the brain and which will enhance vascular and WM remodeling and anti-inflammatory effects and recovery of neurological function after an injury. Our findings that miRNA-126 increases vascular and WM remodeling, as well as promotes angiogenesis and neurite outgrowth, indicate that miR-126 promotes vascular and WM remodeling after stroke in diabetic and non-diabetic brain injury and neurodegenerative disease and thereby improve neurological function after treatment. Some embodiments also comprise our finding that manipulation of miRNA expression in cell-based therapy and cell secretion of EMVs containing miRNA, and such EMVs themselves, may further stimulate endogenous brain cells to generate miR-126 or other miRNAs expression. A significance of our work is that it opens up important and novel ways to understand how exogenously administered cells or miRNA communicate with and alter endogenous brain cells by means of delivery miRNA to activate endogenous restorative events. Neurodegenerative disease, dementia, stroke, neural injury and multiple sclerosis attack millions of Americans annually, and are the most common form of pathology and the leader in loss of quality of life among all diseases. Diabetes mellitus is a severe health problem associated with both microvascular and macrovascular disease, and diabetes complications may include, among other complications, diabetic retinopathy, neuropathy, nephropathy, heart disease and dementia and stroke. Hyperglycemia and diabetes instigate a cascade of events leading to vascular endothelial cell dysfunction, increased vascular permeability, a disequilibrium of angiogenesis (exuberant but dysfunctional neovascularization), and poor recovery after ischemic stroke. Thus, treatment of neurological disease, dementia, injury and diabetes complications with miR-126 or agents that increase miR-126 or miR-126 enriched EMVs may provide an effective therapy for these pervasive neurological insults and diabetes complication.

In accordance with some nonlimiting embodiments, and among other findings, we have discovered unexpectedly that:
Mice with T2DM exhibit decreased miR-126 expression and worse functional outcome after stroke compared to nondiabetic mice. HUCBC treatment of stroke in T2DM mice significantly increased blood serum and ischemic brain tissue miR-126 expression and improves functional outcome after stroke in T2DM mice;
Over-expression of miR-126 in cultured brain endothelial cells increases capillary tube formation and angiogenesis;
Over-expression of miR-126 in cultured brain endothelial cells increases axonal outgrowth when cultured with primary cortical neurons;
Treatment of stroke with D-4F or angiopoietin-1, both increase miR-126 expression as well as improve functional outcome after stroke in non-DM and DM animals;
miR-126 mediates neurological recovery, promotes axonal outgrowth, angiogenesis and mediates the expression of Ang1, a neurovascular restorative agent;
HUCBC promotes neurological recovery via the transmission of miR-126; and
Diabetes decreases BEC miR-126 expression, and release of EMVs, e.g. from HUCBCs, containing high level miR-126 increases BEC miR-126 expression.

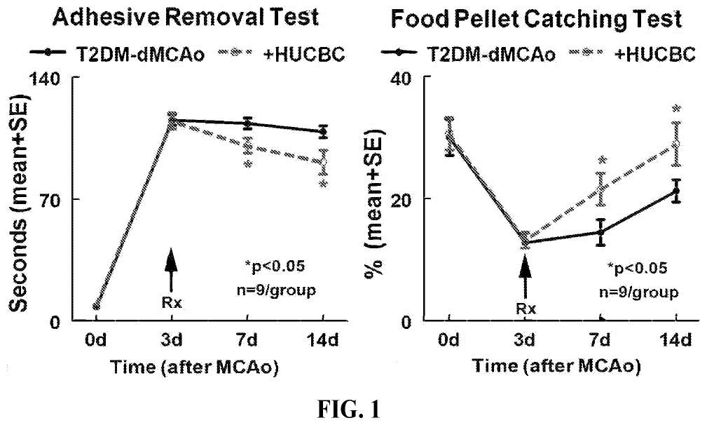

We have found unexpectedly that T2DM mice exhibit decreased miR-126 expression. HUCBC treatment significantly increased blood serum and ischemic brain tissue miR-126 expression. Cg-m+/−FLepr^{db}/J (db/db)-T2DM mice (3 months) were subjected to extraluminal permanent distal MCAo (“dMCAo”) and were randomized to intravenous injection via tail-vein with: 1) phosphate-buffered saline (PBS) control; 2) HUCBC (1×10⁶) at 3 days after dMCAo. Adhesive removal test and food single-pellet reaching test to characterize skilled reaching ability of the stroke-impaired left forepaw were performed 3 days (before treatment) and 7, 14 days after dMCAo by an investigator blinded to the experimental groups. Mice were sacrificed at 14 days after dMCAo. HUCBC treatment of stroke did not decrease lesion volume (T2DM+HUCBC: 12.7±3.0% vs. T2DM-EPBS: 14.5±3.2%), but significantly improves functional outcome at 7 and 14 days after dMCAo compared to T2DM mice (p<0.05). (See FIG. 1).

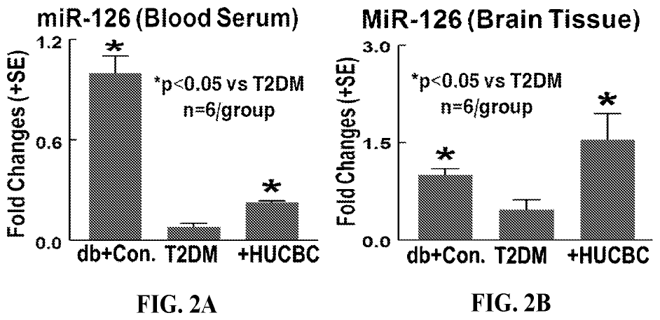

We have found unexpectedly that HUCBC regulates miR-126 expression, Mice (groups and treatment are the same as above in FIG. 1) were sacrificed at 14 days after dMCAo. Blood serum and ischemic brain tissue from the brain ischemic boundary zone (IBZ) were isolated to measure miR-126 expression by TaqMan miRNA assay. FIG. 2A-B show that T2DM mice exhibit significantly decreased miR-126 expression in serum and in the IBZ compared to db+ (no-DM) control mice (p<0.05), while HUCBC treatment in T2DM mice significantly increased miR-126 expression in blood serum and IBZ compared to non-treatment T2DM mice.

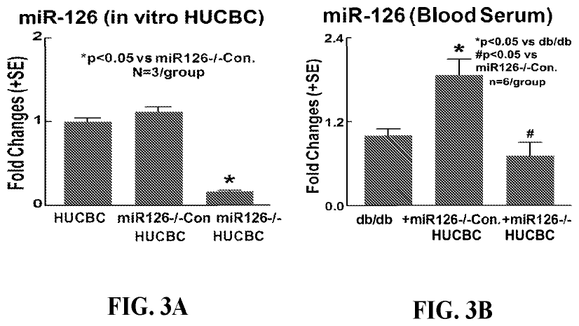

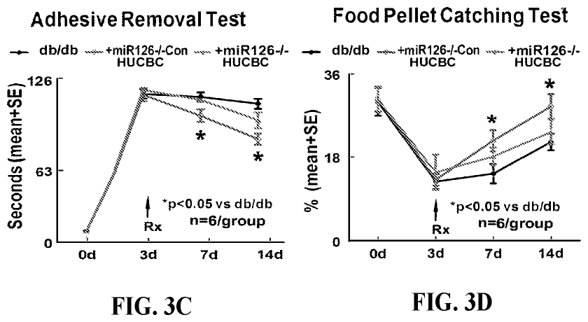

We have found unexpectedly that that knockdown miR-126 attenuates HUCBC-induced neuro-restorative effects after stroke in T2DM mice. To evaluate whether miR-126 mediates HUCBC-induced neurorestorative effects, knockdown of miR-126 in HUCBC (mouse mmu-miR-126-3p inhibitor, miR-126−/−HUCBC) and miR-126 knockdown negative control inhibitor (Thermo Scientific, miR-126−/−Con-HUCBC) was performed using electroporation transfection method. FIG. 3A shows that miR-126−/−HUCBC significantly decreases miR-126 expression compared to miR-126−/−Con-HUCBC and naive HUCBC (p<0.05). Db/db-T2DM mice were subjected to dMCAo and were treated intravenous injection via tail-vein with: 1) PBS; 2) miR-126−/−HUCBC; 3) miR-126−/−Con-HUCBC 3 days after dMCAo. FIG. 3B-D show that miR-126−/−HUCBC treatment of stroke in T2DM significantly decreases miR-126 expression in blood serum and attenuates HUCBC induced functional outcome after stroke in T2DM mice. FIG. 3C shows the time spent to remove the adhesive dots; FIG. 3D shows the proportion of trials in which food pellets are acquired. The data indicated that increasing miR-126 plays an important role in HUCBC-induced neurorestorative effects after stroke.

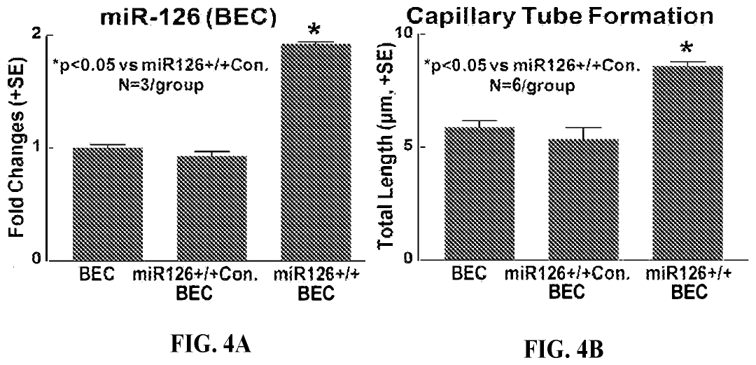

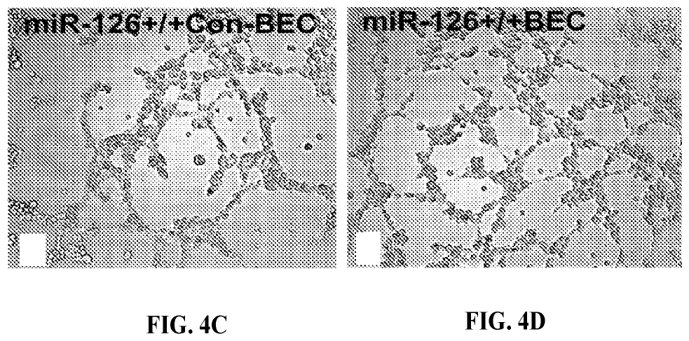

In accordance with some embodiments, without limitation, we have found that miR-126 promotes angiogenesis. To evaluate whether miR-126 regulates vascular remodeling, capillary tube formation in mouse BECs was measured. BECs were transfected with pEGP-mmu-mir-126 Expression vector (MMU-MiR-126 for miR-126 knock-in) or pLenti-III-miR-GFP knock-in Control Vector. (See FIG. 4A-4D). The data show that miR-126+/+BECs significantly increased miR-126 expression compared to knock-in control BECs (miR-126+/+Con-BECs). Then miR-126+/+BECs, miR-126+/+Con-BECs and BEC-control cells were incubated in matrigel for tube formation assay (n=6/group). Total length of capillary tube like formation was quantitated 5 h after culture. The data show that MiR-126+/+BECs significantly increased capillary tube formation compared to miR-126+/+Con-BECs p<0.05). (See FIG. 4A-4D).

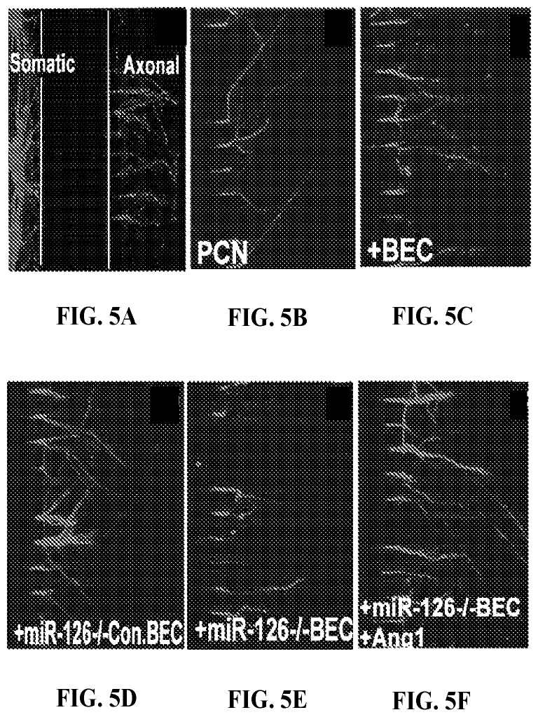

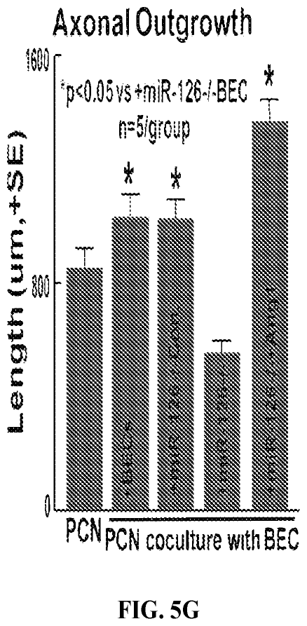

We have found unexpectedly that that miR-126 increases axonal outgrowth. To evaluate whether miR-126 regulates PCN axonal outgrowth, the coculture of BECs with PCNs were performed. PCNs were obtained from pregnant C57BLI6J mice embryos 17 (E17) days old and cultured in vitro. To separate axons from neuronal soma, a microfluidic chamber (Standard Neuron Device) was used. The small dimension of the microgrooves in the chamber allows axons to sprout from the cell-seeded compartment into the other compartment of the chamber, but prevents the passage of cell bodies. BECs were transfected with knockdown of miR-126 (miR-126−/−BEC) or knockdown control (mR-126-I-Con-BEC) and then cocultured with PCNs for 3 days. Then phos-Neurofiliment (SMI-31) immunostaining was performed. The average length of axonal outgrowth of SMI-31 positive cells was measured. (See FIG. 5A-5G). The data show that coculture PCN with miR-126−/−BECs significantly decreases PCN axonal outgrowth compared to when cocultured with the BEC or miR-126−/−Con-BEC group, respectively.

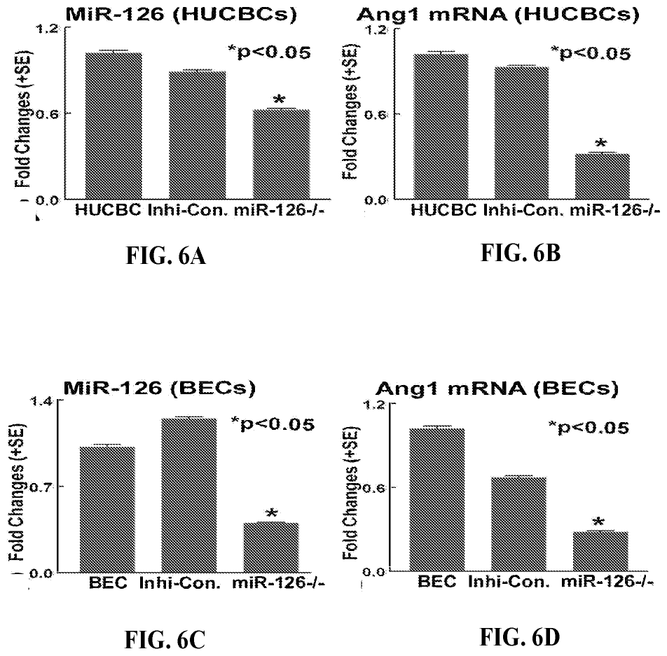

We have found unexpectedly that miR-126 regulates Ang1 expression. To evaluate the effect of miR-126 on Ang1 expression, loss-of-function of miR-126 in HUCBCs or BECs was performed. (See FIG. 6A-6D). The data show that knock-down miR-126 expression in BECs or HUCBC significantly decreased miR-126 expression in HUCBCs (FIG. 6A) and BECs (FIG. 6C) compared to nontransfected control or negative knock-down control, and subsequently decreased Ang1 expression level in HUCBCs (FIG. 6B) and in BECs (FIG. 6D) (n=3/group). These data indicated that manipulation of miR-126 subsequently regulates Ang1 expression. Ang1 treatment significantly attentuates the decreased axonal outgrowth in miR-126−/−BECs group. The data indicated that miR-126 and Ang1 influence PCN axonal outgrowth.

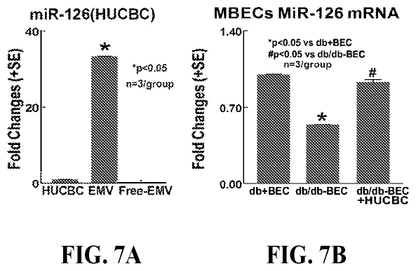

We have found unexpectedly that that diabetes decreases brain endothelial cell miR-126 expression, and HUCBC release of EMVs containing miR-126 increases BEC miR-126 expression. To evaluate whether HUCBC treatment promotes BECs miR-126 expression, HUCBC culture was employed in vitro for 3 days. EMVs were isolated by a series of centrifugations and ultracentrifugations. MiR-126 expression was measured in HUCBC cell lysate and EMVs. FIG. 7A shows that miR-126 derived from HUCBC-EMVs are 30 fold higher than miR-126 from HUCBC cell lysate, and are 40 fold higher than in EMV free supernatant. To evaluate whether diabetes regulates BEC miR-126 expression and whether HUCBC promotes BEC miR-126 expression, BECs were isolated from the IBZ of db+ (no-DM) and db/db (T2DM) mice 3 days after dMCAo. Then transwell coculture model was employed. Db/db-BECs were plated to the lower chamber of the six well plate, with or without HUCBC added in the upper chamber of a Falcon 0.4 pm cell culture insert. FIG. 7B shows that db/db-BECs exhibit significantly decreased miR-126 level compared to db+-BECs, while coculture db/db-BECs with HUCBC significantly increases miR-126 expression in db/db-BECs compared to db/db-BECs culture alone. These data indicate that HUCBCs release high levels of miR-126 into EMVs. EMVs secreted from HUCBC subsequently increase BEC expression of miR-126.

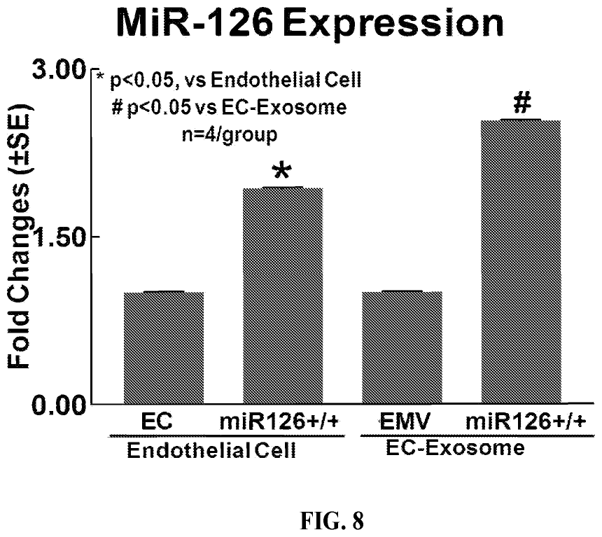

We have found unexpectedly that endothelial cells over-expressing miR-126 increases EMV miR-126 expression. To evaluate whether endothelial cells (“ECs”) secrete EMVs containing miR-126, mouse brain ECs were transfected with pEGP-mmu-mir-126 Expression vector (MMU-MiR-126 for miR-126 knock-in) or pLenti-III-miR-GFP knock-in Control Vector as control. Then ECs and EMVs were isolated to measure miR-126 expression. The data (FIG. 8) show that knock-in miR-126 in ECs (miR-126+/+-ECs) not only increases EC miR-126 expression, but also significantly increases EMV miR-126 expression compared to control, respectively.

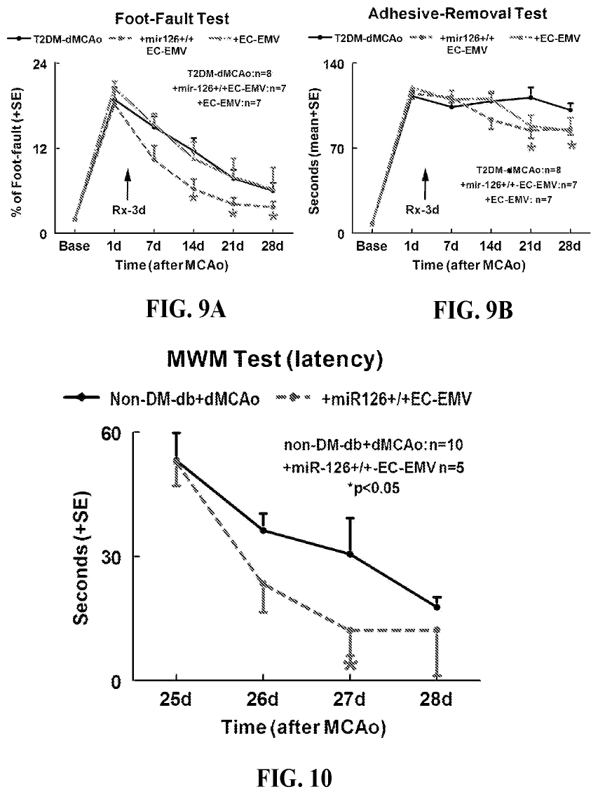

We have found unexpectedly that overexpression miR-126 EMV treatment of stroke significantly improves neurological functional outcome in T2DM mice. To evaluate whether miR-126 regulates neurological functional outcome after stroke in T2DM animals, EMV was isolated from EC control or miR-126 overexpressing endothelial cells (miR-126+/+-ECs). BKS.Cg-m+/+Lepr^{db}/J (db/db) T2DM mice were subjected to distal MCAo (dMCAo) and treatment was initiated 3 days after stroke via tail vine injection with: 1) PBS as control (n=8); 2) EMV derived from endothelial cell (EC-EMV, 20 μg per mouse, n=7); 3) EMV derived from miR-126 over-expressing endothelial cells (miR-126+/+-EC-EMV, 20μg per mouse, n=7). A battery of functional tests were performed. The data (FIG. 9A-B) show that EC-EMV marginally improves adhesive removal function at 21 days after stroke compared to non-treatment control (p=0.052), while miR-126+/+-EC-EMV significantly improves neurological functional outcome measured by Foot-fault and adhesive removal tests compared to non-treatment controls (p<0.05). The data indicate that miR-126+/+-EC-EMV treatment improves functional outcome after stroke in T2DM mice.

We have found unexpectedly that overexpression miR-126 in EC-EMV treatment of stroke significantly improves cognitive functional outcome in non-diabetic mice. To evaluate whether miR-126 regulates cognitive functional outcome after stroke, EMV was isolated from EC control or miR-126+/+-ECs. Non-DM db+ mice were subjected to distal MCAo (dMCAo) and treatment was initiated 1 day after stroke via tail vine injection with: 1) PBS as control (n=10); 2) miR-126+/+-EC-EMV (20 μg per mouse, n=5). Morris Water Maze (MWM) cognitive functional test was performed. The data (FIG. 10) show that miR-126+/+-EC-EMV significantly improves cognitive functional outcome compared to non-treatment controls (p<0.05). The data indicated that miR-126+/+-EC-EMV treatment improves cognitive functional outcome after stroke.

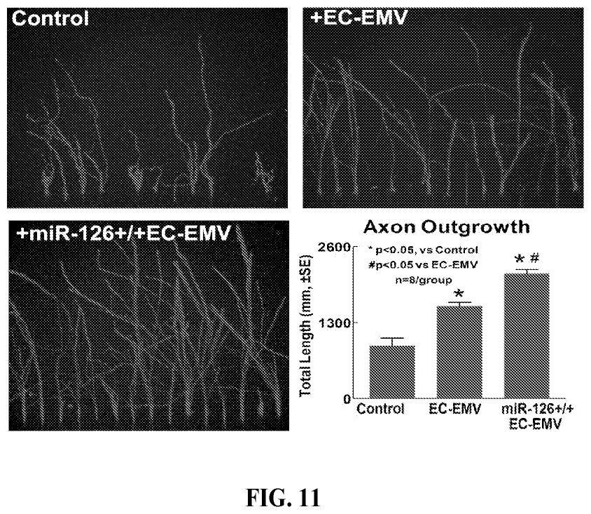

We have found unexpectedly that miR-126+/+-EC-EMV increases primary cortical neuron axonal outgrowth. To evaluate whether miR-126 regulates PCN axonal outgrowth, PCNs were obtained from pregnant C57BL/6J mouse embryos 17 (E17) days old and cultured in vitro. To separate axons from neuronal soma, a microfluidic chamber (Standard Neuron Device) was used. The small dimension of the microgrooves in the chamber allows axons to sprout from the cell-seeded compartment into the adjacent compartment of the chamber, but prevents the passage of cell bodies. The PCN were treated with: 1) non-treatment control; 2) EC-EMV (10 ng/ml); 3) miR-126+/+-EC-EMV (10 ng/ml) for 3 days. The cells were then immunostained for phos-Neurofiliment (SMI-31). The average length of axonal outgrowth of phos-Neurofiliment positive axons were measured. The data (FIG. 11) shows that EC-EMV and miR-126+/+-EC-EMV both significantly increased PCN axonal outgrowth compared to non-treatment control, while miR-126+/+-EC-EMV significantly increased outgrowth compared to EC-EMV alone (p<0.05). The data indicated that miR-126 increase PCN axonal outgrowth.

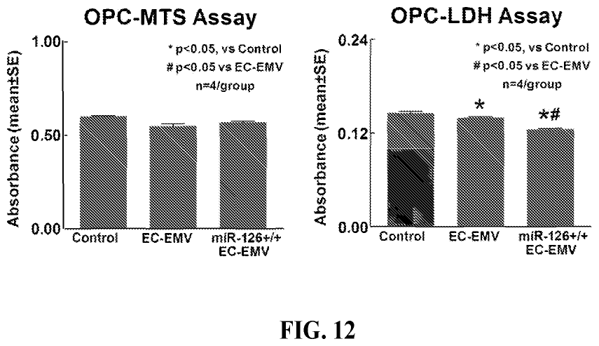

We have found unexpectedly that miR-126 increases oligodendrocyte precursor cell (OPC) survival. To evaluate whether miR-126 regulates OPC survival; an immortalized mouse premature OL cell line (N20.1, generously provided by Dr. Anthony Campagnoni, University of California) was used. OPCs were subjected to 2 h OGD then treated with: 1) non-treatment control; 2) EC-EMV (10 ng/ml); 3) miR-126+/+-EC-EMV ((10 ng/ml) for 48 hr. Lactate dehydrogenase (LDH, for cell death) and cell proliferation assay (MTS ([3-(4,5-dimethylthiazol-2-yl)-5-(3 -carboxymethoxyphenyl)-2-(4-sulfophenyl)-2H-tetrazolium), Promega) were employed. The data shows that EC-EMV and miR-126+/+EC-EMV both significantly increase OPC survival, but did not regulate OPC proliferation compared to non-treatment control (p<0.05) (FIG 12), while miR-126+/+-EC-EMV has higher OPC cell survival compared to EC-EMV alone (p<0.05). These data suggest that miR-126 increases OPC survival.

In some nonlimiting embodiments, miR-126, or agents which increase miR-126, or agents which deliver miR-126, such as miR-126 enriched EMVs, derived from cells, e.g. HUCBCs, or other sources, may be used as therapies to promote neurological function after stroke in the diabetes and non-diabetes population, neural injury, multiple sclerosis and neurodegenerative disease and diabetes complications. Among other findings, we found that miR-126 significantly increases angiogenesis and neurite outgrowth. Thus, in accordance with some nonlimiting embodiments, miR-126 or related agents or cell-based therapy or miR-126 enriched EMVs which increase miR-126 may be administered to patients before or after the onset of injury or disease to reduce the neurological deficits associated with disease and possibly aging and diabetes complications.

Some embodiments comprise the use of miR-126 or increasing miR-126 related agents or cell-based therapy or miR-126 enriched EMVs to improve neurological function and treat diabetes complications. To our knowledge, it has not been reported that these agents have the property of increasing WM remodeling and improving neurological outcome post-stroke, neural injury and neurodegenerative disease and diabetes complications.

Thus, without limitation and without waiver or disclaimer of any embodiments or subject matter, some embodiments comprise microRNA-126, a promoter of microRNA-126 expression, a microRNA-126 mimic, such as human umbilical cord blood cells and endothelial cells, and endothelial progenitor cells (“EPC”) (as nonlimiting examples), and microRNA-126-enriched EMVs (all for the foregoing collectively “miRNA126 agent(s)”) to prevent, control, or alleviate mammalian illness or injury through the selective application of such miRNAs. In accordance with some embodiments, without limitation, one may inhibit such illness or injury through miRNA-126 agent administration for a finite interval of time, thereby limiting the development or course of such illness or injury.

In accordance with some embodiments, there is a high likelihood that the duration of therapy comprising miRNA-126 agent administration would be relatively brief and with a high probability of success. Prophylactic miRNA-126 agent administration of some embodiments may greatly reduce the incidence of damage associated with many forms of illness or injury.

Any appropriate routes of miRNA-126 agent administration known to those of ordinary skill in the art may comprise embodiments of the invention.

MiRNA-126 agents of some embodiments would be administered and dosed in accordance with good medical practice, taking into account the clinical condition of the individual patient, the site and method of administration, scheduling of administration, patient age, sex, body weight and other factors known to medical practitioners. The “pharmaceutically effective amount” for purposes herein is thus determined by such considerations as are known in the art. The amount must be effective to achieve improvement, including but not limited to, decreased damage or injury, or improvement or elimination of symptoms and other indicators as are selected as appropriate measures by those skilled in the art.

In accordance with some embodiments, miRNA-126 agents can be administered in various ways. They can be administered alone or as an active ingredient in combination with pharmaceutically acceptable carriers, diluents, adjuvants and vehicles. The miRNA-126 agents can be administered orally, subcutaneously or parenterally including intravenous, intraarterial, intramuscular, intraperitoneal, and intranasal administration as well as intrathecal and infusion techniques, or by local administration or direct inoculation to the site of disease or pathological condition. Implants of the miRNA-126 agents may also be useful. The patient being treated is a warm-blooded animal and, in particular, mammals including humans. The pharmaceutically acceptable carriers, diluents, adjuvants and vehicles as well as implant carriers generally refer to inert, non-toxic solid or liquid fillers, diluents or encapsulating material not reacting with the active components of the invention. In some embodiments, miRNA-126 agents may be altered by use of antibodies to cell surface proteins to specifically target tissues of interest.

Since the use of miRNA-126 agent administration in accordance with some embodiments specifically targets the evolution, expression, or course of associated pathologies, it is expected that the timing and duration of treatment in humans may approximate those established for animal models in some cases. Similarly, the doses established for achieving desired effects using such compounds in animal models, or for other clinical applications, might be expected to be applicable in this context as well. It is noted that humans are treated generally longer than the experimental animals exemplified herein which treatment has a length proportional to the length of the disease process and drug effectiveness. The doses may be single doses or multiple doses over periods of time. The treatment generally has a length proportional to the length of the disease process and drug effectiveness and the patient species being treated.

When administering the miRNA-126 agents of some embodiments parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

When necessary, proper fluidity can be maintained, for example, by the use of a coating such as lecithin, by the maintenance of the required size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for such miRNA-126 agent compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to some embodiments of the present invention, however, any vehicle, diluent, or additive used would have to be compatible with the miRNA-126 agents.

Sterile injectable solutions can be prepared by incorporating miRNA-126 agents utilized in practicing the some embodiments of the present invention in the required amount of the appropriate solvent with various of the other ingredients, as desired.

A pharmacological formulation of some embodiments may be administered to the patient in an injectable formulation containing any compatible carrier, such as various vehicle, adjuvants, additives, and diluents; or the inhibitor(s) utilized in some embodiments may be administered parenterally to the patient in the form of slow-release subcutaneous implants or targeted delivery systems such as monoclonal antibodies, vectored delivery, iontophoretic, polymer matrices, liposomes, and microspheres. Many other such implants, delivery systems, and modules are well known to those skilled in the art.

In some embodiments, without limitation, the miRNA-126 agents may be administered initially by intravenous injection to bring blood levels to a suitable level. The patient's levels are then maintained by an oral dosage form, although other forms of administration, dependent upon the patient's condition and as indicated above, can be used. The quantity to be administered and timing of administration may vary for the patient being treated.

Additionally, in some embodiments, without limitation, miRNA-126 agents may be administered in situ to bring internal levels to a suitable level. The patient's levels are then maintained as appropriate in accordance with good medical practice by appropriate forms of administration, dependent upon the patient's condition. The quantity to be administered and timing of administration may vary for the patient being treated.

While some embodiments have been particularly shown and described with reference to the foregoing preferred and alternative embodiments, it should be understood by those skilled in the art that various alternatives to the embodiments described herein may be employed in practicing the invention without departing from the spirit and scope of the invention as defined in the following claims. It is intended that the following claims define the scope of the invention and that the methods, systems, and compositions within the scope of these claims and their equivalents be covered thereby. This description of some embodiments should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

What is claimed is:

1. A method comprising administering a composition comprising a pharmaceutically effective amount of one or more agents selected from the group consisting of microRNA-126 (miR-126), a promoter of miR-126 expression, a miR-126 mimic and isolated Exosomes/Microvesicles containing miR-126 to a patient diagnosed with a condition selected from the group consisting of: stroke, brain injury, dementia, multiple sclerosis, and diabetes mellitus type 2, to promote, increase, and/or improve neurological function or neurological recovery in the patient.

2. The method of claim 1, wherein the patient is diagnosed with a stroke.

3. The method of claim 2, wherein the patient has been diagnosed with Type II diabetes.

4. The method according to claim 1, wherein the patient is administered a therapeutically effective amount of isolated Exosomes/Microvesicles containing miR-126.

5. The method according to claim 1, wherein improvement of neurological function or neurological activity in the patient is associated with any one or more of: increased vascular remodeling, increased white matter remodeling, increased angiogenesis, increased neurite outgrowth, increased axonal outgrowth, and oligodendrocyte survival.

6. The method according to claim 1, wherein the one or more agents are administered intravenously.

7. The method according to claim 4, wherein the isolated Exosomes/Microvesicles containing miR-126 are isolated from human umbilical cord blood cells (HUCBC).

8. The method according to claim 4, wherein the isolated Exosomes/Microvesicles containing miR-126 are isolated from brain endothelial cells (BEC), or BEC modified to increase expression of miR-126.

9. The method according to claim 4, wherein the isolated Exosomes/Microvesicles containing miR-126 are isolated from any cell source which contain miR-126 and are enriched with miR-126.

10. The method of claim 1, wherein the patient is a human patient.

11. The method of claim 1, wherein miR-126 comprises the nucleotide sequence of SEQ ID NO: 1.
